(19) United States
(12) Patent Application Publication
(10) Pub. No.: US 2004/0115837 A1
(43) Pub. Date: Jun. 17, 2004

(54) Methods of measuring the dissolution rate of an analyte in a non-aqueous liquid composition

(76) Inventors: Randal Lee Schapaugh, Battle Creek, MI (US); Edward L. Ciolkowski, Galesburg, MI (US); Leslie C. Eaton, Schoolcraft, MI (US)

Correspondence Address: PHARMACIA & UPJOHN, 301 HENRIETTA ST, 0228-32-LAW, KALAMAZOO, MI, 49007, US

(21) Appl. No.: 10/658,164

(22) Filed: Sep. 9, 2003

Related U.S. Application Data
(60) Provisional application No. 60/429,260, filed on Nov. 27, 2002.

(51) Int. Cl. G01N 33/536

(52) U.S. Cl. 436/536

(57) ABSTRACT

The present invention provides a method of measuring the dissolution rate of an analyte in a non-aqueous liquid composition and in particular to in vitro methods for measuring the dissolution rate of a drug in a sustained release dosage form.

### CROSS REFERENCE TO RELATED APPLICATION

This application claims the benefit of U.S. Provisional Application Serial No:60/429,260 filed 27 Nov. 2002, under 35 USC 119(e)(i), which is incorporated herein by reference in its entirety.

### FIELD OF THE INVENTION

The present invention refers to a method of characterizing the transfer of an analyte from a non-aqueous liquid composition to an aqueous medium and in particular to an in vitro method for measuring the dissolution of a drug from a sustained release dosage form.

### BACKGROUND OF THE INVENTION

One important aspect of formulating pharmaceutical compositions is the drug's pharmacokinetic behavior. Depending on a variety of factors, such as the physical state of the drug (i.e. gas, liquid, solid), its crystal form, its particle size, the dosage form, and the excipients used, the time-dependent release of the drug in the body can vary drastically. Even if the same drug is presented in the same dosage form, lot-to-lot variations can occur.

For regulatory approval pharmacokinetic behavior is often determined by administering the drug to animals or humans and measuring the amount of drug or its metabolites in blood at certain points of time after administration. This method is time-consuming and expensive and is generally not employed to control the quality of the pharmaceuticals during the manufacturing process. A number of methods have been devised to assess the in vivo pharmacokinetic behavior of drugs in in vitro tests. Some of the tests have been standardized and are described e.g. in the United States Pharmacopeia (USP). Commonly used USP methods are the basket method (USP method I) and the paddle method (USP method II). In addition, to these standardized methods a large number of methods for specific individual applications have been described. An overview over a number of dissolution methods can be found e.g. in G. K. Shiu, Drug Information Journal, 30, 1045-1054, (1996).

Andonaegui et al. (Drug Development and Industrial Pharmacy, 25(11), 1199-1203 (1999)) describe an in vitro method for predicting the in vivo performance of high-fat diet. The dissolution profiles of theophylline in three types of sustained-release matrix tablets were investigated. To improve the in vitro/in vivo-correlation for a high-fat diet the tablets were pretreated by mixing with peanut oil before the dissolution testing in the USP paddle test.

Japanese patent application JP 05-249097 describes a dissolution test for predicting the in vivo release of a sustained-release tablet. The tablet is subjected to the paddle method, taken out, treated with oils and fats and then either returned to the paddle apparatus together with beads in the aqueous dissolution medium or submerged in a basket. This method is said to predict the concentration of a drug in blood plasma inside a living body without being affected by the release control mechanism of the sustained release tablet.

Various in vitro dissolution methods for microparticulate drug delivery systems are compared by Conti et al. in Drug Development and Industrial Pharmacy, 21(10), 1233-1233 (1995). The influences of stirring speed, ionic strength and the presence of a surfactant are investigated.

Dissolution methods for testing oily drug preparations have also been described. Takahashi et al. (Chem. Pharm. Bull., 42(8), 1672-1675, (1994)) compare the paddle method and the rotating dialysis cell method. In a variation of the rotating dialysis cell method, octanol was employed as external phase, while an acidic solution was used as an internal phase.

Machida et al. (Chem. Pharm. Bull., 34(6), 2637-2641, (1986)) describe one attempt to overcome the problems encountered in measuring the dissolution characteristics of oily drug preparations. They propose using a modification of the paddle method of the Japanese Pharmacopeia method 2 with an additional assistant wing to stir the surface of the aqueous dissolution medium. Furthermore, beads were added to improve agitation and a bile salts solution was employed as the aqueous dissolution medium.

The pharmacokinetic behavior of non-aqueous pharmaceutical compositions, in which the drug is dissolved or dispersed in a non-aqueous base, is difficult to predict reliably using prior art methods. The precision and reliability of the in vitro measurements is often low and the results of the in vitro measurements do not always correlate with the behavior of the drug in vivo. Therefore, one object of the present invention is to provide a reliable method, with which the dissolution rate of an analyte in a non-aqueous liquid composition can be determined. A further object of the present invention is to provide a respective method which employs a standardized dissolution apparatus.

### SHORT DESCRIPTION OF THE FIGURES

FIG. 1 shows one possibility of plotting the dissolution rate if the amount of analyte is determined more than once.

FIG. 2 shows a further possibility of plotting the dissolution rate if the amount of analyte is determined more than once.

FIG. 3 shows a scheme of a typical paddle assembly. The drawing is not to scale.

FIG. 4 shows the variations in spreading behavior observed in example 1.

FIG. 5 shows the relationship between the duration of sustained release in vivo with the in vitro paddle method, if the non-aqueous liquid composition is not diluted with a non-aqueous diluent in the paddle method.

FIG. 6 shows the relationship between the duration of sustained release in vivo with the in vitro paddle method, if the non-aqueous liquid composition is diluted with a non-aqueous diluent in the paddle method.

FIG. 7 illustrates the effect of the size of the aliquot on the dissolution rate.

FIG. 8 shows the linearity of the method of the present invention.

### SUMMARY OF THE INVENTION

In one embodiment the present invention provides a method of determining the dissolution rate of an analyte in a non-aqueous liquid composition, comprising the steps of:

(a) providing a non-aqueous liquid composition comprising an analyte and a non-aqueous base;

(b) adding a non-aqueous diluent to the non-aqueous liquid composition to provide a diluted non-aqueous liquid composition;

(c) introducing at least part of the diluted non-aqueous liquid composition and an aqueous dissolution medium into a dissolution testing apparatus;

(d) contacting the diluted non-aqueous liquid composition and the aqueous dissolution medium for a predetermined time; and

(e) determining the amount of analyte in the aqueous dissolution medium.

In a further embodiment of the invention provides a method of determining the dissolution rate of an analyte in a non-aqueous liquid composition, comprising the steps of:

(a) providing a non-aqueous liquid composition comprising an analyte and a non-aqueous base;

(b) introducing at least part of the diluted non-aqueous liquid composition and an aqueous dissolution medium into a dissolution testing apparatus, wherein the aqueous dissolution medium comprises a buffer having a molarity of from about 0.1 mM to about 10 mM;

(c) contacting the non-aqueous liquid composition and the aqueous dissolution medium for a predetermined time; and

(d) determining the amount of analyte in the aqueous dissolution medium.

A method of determining the dissolution rate of an analyte in a non-aqueous liquid composition is also disclosed, which comprises the steps of:

(a) providing a non-aqueous liquid composition comprising an analyte and a non-aqueous base;

(b) introducing at least part of the diluted non-aqueous liquid composition and an aqueous dissolution medium into a dissolution testing apparatus, wherein the volume ratio of non-aqueous liquid composition to aqueous dissolution medium in the dissolution testing apparatus is from about 1:2,000 to about 1:100,000;

(c) contacting the non-aqueous liquid composition and the aqueous dissolution medium for a predetermined time; and

(d) determining the amount of analyte in the aqueous dissolution medium.

### DETAILED DESCRIPTION OF THE INVENTION

The present invention provides reliable methods for determining the dissolution rate of an analyte in a non-aqueous liquid composition. Although the methods are preferably employed to determine the dissolution rate of a pharmaceutically active ingredient from a pharmaceutical composition, they can also be employed in other fields of analytical chemistry, e.g. to determine the rate with which contaminants are leached from oils into the environment, to determine the rate with which active agents such as corrosion inhibitors and the like are depleted from oily bases or to measure the rate with which components are released from pesticides or fertilizers.

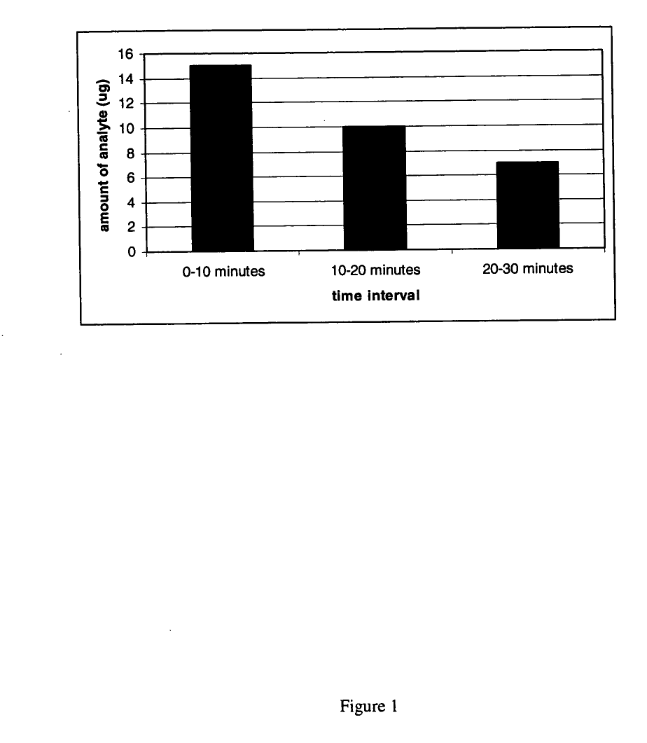

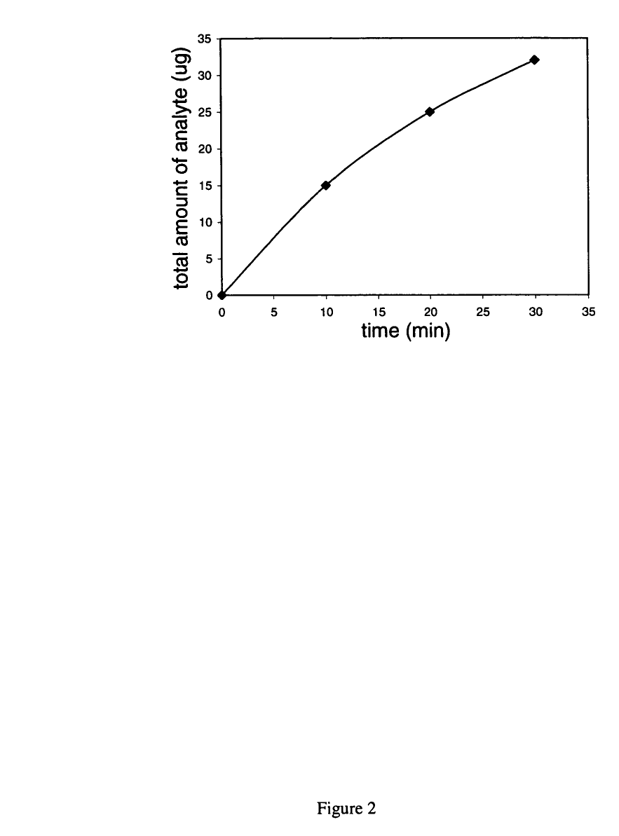

The term “dissolution rate” is the rate with which the analyte dissolves in the non-aqueous dissolution medium. If the amount of analyte in the aqueous dissolution medium is determined at only one predetermined time, the dissolution rate is the total amount of analyte, which has been dissolved up to that predetermined time (e.g. expressed in weight) divided by the predetermined time. For example, if it is determined that 3 μg of analyte have been dissolved after 30 minutes, the dissolution rate would be 3 μg/30 minutes, or 0.1 μg/minute. If the amount of analyte in the aqueous dissolution medium is determined more than one time, then the dissolution rate can be illustrated in several different ways, which are known in the art. One common way is to plot the data in a two-dimensional graph, in which the x-axis represents the time line and the y-axis represents the amount of analyte dissolved between the nth and the (n-1)th analysis of the aqueous dissolution medium. A further common way is to plot the data in a two-dimensional graph, in which the x-axis is again a time line and the y-axis represents the total amount of analyte dissolved between beginning of the measurement and the nth analysis of the aqueous dissolution medium. Of course the same information can be presented in a table or any other suitable form other than the two-dimensional graphs discussed above. The following series of experiments can be used as an example: a non-aqueous liquid composition is investigated and the amount of analyte dissolved is determined at 10 minutes (n=1), 20 minutes (n=2), and 30 minutes (n=3). After 10 minutes 15 μg of analyte have dissolved, after 20 minutes 25 μg of analyte have dissolved and after 30 minutes 32 μg of analyte, in total, have dissolved. In the first case the plot as shown in FIG. 1 would be obtained, while in the second case the plot would be as in FIG. 2.

As used herein the term “non-aqueous liquid composition” is any composition which is liquid at the contacting temperature and which comprises an analyte and a non-aqueous base. The mixture of the analyte and the non-aqueous base can be in any form, for example they can form a solution, an emulsion or suspension. If the analyte is suspended in the non-aqueous base, the particle size of the analyte will generally be in the range of from about 50 nm to about 200 microns, preferably from about 100 nm to about 200 microns. The concentration of the analyte in the non-aqueous liquid composition is not particularly restricted.

The non-aqueous liquid composition is preferably a pharmaceutical composition. In the methods of the present invention the pharmaceutical composition will generally be a liquid suitable for parenteral, oral, sublingual, intranasal, intrabronchial, pulmonary, intramammary, rectal, vaginal, ocular, or topical application. However, it is also possible to determine the dissolution rate of an analyte in a pharmaceutical composition where the pharmaceutical composition is contained in a capsule. In this case, the shell of the capsule will disintegrate on contact with the aqueous dissolution medium and release its contents.

The term “analyte” refers to a component in a non-aqueous liquid composition the dissolution of which component is to be characterized. The analyte can be any component in the composition. Examples of analytes are, but are not restricted to, a contaminant, an active component, or an inactive component. In the case of pharmaceutical compositions the analyte will typically be the pharmaceutically active ingredient; but it can also be an excipient or any other component of the pharmaceutical composition. The method of the present invention is not restricted to the determination of a single analyte; if desired two or more analytes can be determined. The method of the invention is not restricted to the determination of analytes with any particular physical or chemical characteristics. Virtually any analyte—organic or inorganic— can be determined with the method of the invention so long as the analyte is at least partially soluble in the aqueous dissolution medium chosen for the method. Examples of analytes, which can be determined using the method of the invention include the following illustrative, non-limiting classes: ACE inhibitors; α-adrenergic agonists; β-adrenergic agonists; α-adrenergic blockers; β-adrenergic blockers (beta blockers); alcohol deterrents; aldose reductase inhibitors; aldosterone antagonists; amino acids; anabolics; analgesics (both narcotic and non-narcotic); anesthetics; anorexics; antacids; anthelmintics; antiacne agents; antiallergics; antiandrogens; antianginal agents; antianxiety agents; antiarrythmics; antiasthmatics; antibacterial agents and antibiotics; antialopecia and antibaldness agents; antiamebics; antibodies; anticholinergic drugs; anticoagulants and blood thinners; anticolitis drugs; anticonvulsants; anticystitis drugs; antidepressants; antidiabetic agents; antidiarrheals; antidiuretics; antidotes; antiemetics; antiestrogens; antiflatulents; antifungal agents; antigens; antiglaucoma agents; antihistaminics; antihyperactives; antihyperlipoproteinemics; antihypertensives; antihyperthyroid agents; antihypotensives; antihypothyroid agents; anti-infectives; anti-inflammatories (both steroidal and nonsteroidal); antimalarial agents; antimigraine agents; antineoplastics; antiobesity agents; antiparkinsonian agents and antidyskinetics; antipneumonia agents; antiprotozoal agents; antipruritics; antipsoriatics; antipsychotics; antipyretics; antirheumatics; anti secretory agents; anti-shock medications; antispasmodics; antithrombotics, antitumor agents; antitussives; antiulceratives; antiviral agents; anxiolytics; bactericidins; bone densifiers; bronchodilators; calcium channel blockers; carbonic anhydrase inhibitors; cardiotonics and heart stimulants; chemotherapeutics; choleretics; cholinergics; chronic fatigue syndrome medications; CNS stimulants; coagulants; contraceptives; cystic fibrosis medications; decongestants; diuretics; dopamine receptor agonists; dopamine receptor antagonists; enzymes; estrogens; expectorants; gastric hyperactivity medications; glucocorticoids; hemostatics; HMG CoA reductase inhibitors; hormones; hypnotics; immunomodulators; immunosuppressants; laxatives; medicaments for oral and periodontal diseases; miotics; monoamine oxidase inhibitors; mucolytics; multiple sclerosis medications; muscle relaxants; mydriatics; narcotic antagonists; NMDA receptor antagonists; oligonucleotides; ophthalmic drugs; oxytocics; peptides, polypeptides and proteins; polysaccharides; progestogens; prostaglandins; protease inhibitors; respiratory stimulants; sedatives; serotonin uptake inhibitors; sex hormones including androgens; smoking cessation drugs; smooth muscle relaxants; smooth muscle stimulants; thrombolytics; tranquilizers; urinary acidifiers; urinary incontinence medications; vasodilators; vasoprotectants; and combinations thereof.

It will be understood that any reference herein to a particular drug compound includes tautomers, stereoisomers, enantiomers salts and prodrugs of that compound and is not specific to any one solid-state form of the drug.

The method of the invention is especially suitable for determining the dissolution rate of cephalosporins such as third generation cephalosporins. Examples thereof are, but are not limited to, ceftiofur, cefepime, cefixime, cefoperazone, cefotaxime, cefpodoxime, ceftazidime, ceftizoxime, ceftriaxone, moxalactam, pharmaceutically acceptable salts and derivatives thereof. A particularly preferred cephalosporin is ceftiofur, pharmaceutically acceptable salts and derivatives thereof.

Ceftiofur is presently commercially available from Pharmacia under the trade designations Naxel® and Excenel®. Another preferred form of ceftiofur is ceftiofur crystalline free acid (CCFA). This compound as well as pharmaceutical formulations thereof are described in U.S. Pat. No. 5,721,359, which is incorporated herein in its entirety.

The non-aqueous liquid composition also contains a non-aqueous base, which is typically liquid at the contacting temperature and may be miscible, partially immiscible, or immiscible with water. The non-aqueous base can be a lipid or mixture of lipids, such as fats, waxes, and sterols. The lipid can be hydrogenated or non-hydrogenated, saturated, unsaturated, or polyunsaturated, and may be further modified by techniques commonly known in the art. It is preferred that the non-aqueous base is selected from waxes or fats, either natural or synthetic. As used herein, the term “Waxes” refers to mixtures of esters of long—chain carboxylic acids with long—chain alcohols. The carboxylic acid in a wax typically has an even number of carbons from 16 through 36 and while the alcohol usually has an even number of carbons from 24 through 36. As used herein, the term “fats” refers to esters of long chain carboxylic acids and the triol glycerol, which can be natural or synthetic, and Fats can be liquid, solid, or semi-solid at room temperature (about 25 degree C.). “Fats” are also called glycerides, triacylglycerols, and triglycerides. A fat that is liquid at room temperature is also called “oil.” Thus, as used herein, the term “fat” encompasses “oil.” In the present invention, it is more preferred that the non-aqueous base is a natural or synthetic oil.

Illustrative examples of synthetic oils suitable as the non-aqueous base include triglycerides, or propylene glycol di-esters of saturated or unsaturated fatty acids having from 6 to 24 carbon atoms. Such carboxylic acids are meant to comprise those carboxylic acids having from 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, eicosanoic, heneicosanoic, docosanoic and lignoceric acid. Examples of unsaturated carboxylic acids include oleic, linoleic, linolenic acid and the like. It is understood that the tri-glyceride vehicle may include the mono-, di-, or triglyceryl ester of the fatty acids or mixed glycerides and/or propylene glycol di-esters wherein at least one molecule of glycerol has been esterified with fatty acids of varying carbon atom length. The following are examples of triglyceryl esters: tri-unsaturated esters including triolein, trilinolein and trilinolenin; saturated tri-saturated esters including tripalmitin, tristearin, and tridecanoin. Further examples of triglyceryl esters include di-saturated-mono-unsaturated types: oleodisaturated esters such as 1,2-dipalmitoyl-3-oleoyl-rac-glycerol or 1,3-dipalmitoyl-2-oleoyl-rac-glycerol; linoleodisaturated esters such as 1,3-dipalmitoyl-2-linoleoyl-rac-glycerol. Further examples of triglycerides are mono-saturated-di-unsaturated esters: such as monosaturated-oleolinolein esters including 1-Palmitoyl-2-oleoyl-3-linoleoyl-rac-glycerol and 1-linoleoyl-2-oleoyi-3-stearoyl-rac-glycerol, and mono-saturated-dilinolein esters including 1,2-dilinoleoyl-3-palmitoyl-rac-glycerol.

Examples of diglyceril esters include: the di-unsaturated esters such as 1,2-diolein or 1,3-diolein, 1,2-dilinolein or 1,3-dilinolein and 1,2-dilinolenin or 1,3-dilinolenin; saturated di-saturated esters such as 1,2-dipalmitin or 1,3-dipalmitin, 1,2-distearin or 1,3-distearin, and 1,2-didecanoin or 1,3-didecanoin; saturated-unsaturated diglyceril esters such as 1-palmitoyl-2-oleoyl-glycerol or 1-oleoyl-2-palmitoyl-glycerol, 1-palmitoyl-2-linoleoyl-glycerol or 1-linoleoyl-2-palmitoyl-glycerol.

Examples of monoglyceril esters include: unsaturated esters such as 1-olein or 2-olein, 1-linolein or 2-linolein and 1-linolenin or 2-linolenin; saturated esters such as 1-palmitin or 2-palmitin, 1-stearin or 2-stearin, and 1-decanoin or 2-decanoin.

Examples of polyethylene glycol (PEG) di-esters include: di-unsaturated esters such as 1,2-diolein or 1,3-diolein, 1,2-dilinolein or 1,3-dilinolein and 1,2-dilinolenin or 1,3-dilinolenin; saturated di-saturated esters such as 1,2-dipalmitin or 1,3-dipalmitin, 1,2-distearin or 1,3-distearin, and 1,2-didecanoin or 1,3-didecanoin. Further examples of PEG di-esters from saturated-unsaturated diglyceril esters include: 1-palmitoyl-2-oleoyl-glycerol or 1-oleoyl-2-palmitoyl-glycerol, 1-palmitoyl-2-linoleoyl-glycerol or 1-linoleoyl-2-palmitoyl-glycerol.

Illustrative examples of natural oils are canola oil, coconut oil, corn oil, peanut oil, sesame oil, olive oil, palm oil, safflower oil, soybean oil, cottonseed oil, rapeseed oil, sunflower oil and mixtures thereof. Of these cottonseed oil is preferred.

The non-aqueous base may be modified by means known in the art. For example, in embodiments using a peroxidized unsaturated oil base, modified base may have a peroxide value of between about 0.1 and about 600, and in some embodiments about 10, about 20, about 40, or about 80 or any value in between. As used herein, peroxide values are expressed as milliequivalents (mEq) of peroxide per 1000 grams of oil sample.

Apart from the above-mentioned components the non-aqueous liquid composition can also contain additional compounds. For example, if the non-aqueous liquid composition is a pharmaceutical composition, it can contain any pharmaceutically acceptable components. Typical additional components are, for example, pharmaceutically active ingredients, excipients, additives, suspending agents, preservatives, wetting agents, thickeners, buffers and flocculating agents. Suspending agents, such as gums (e.g., acacia, carrageenan, sodium alginate and tragacanth), cellulosics (e.g., sodium carboxymethylcellulose, microcrystalline cellulose, and hydroxyethylcellulose), and clays (e.g., bentonite and colloidal magnesium aluminum) may be included. Preservatives, such as methyl and propyl paraben, benzyl alcohol, chlorobutanol and thimerosal may be added. Anionic surfactants (e.g., docusate sodium and sodium lauryl sulfate), nonionic surfactants (e.g. polysorbates, polyoxamers, octoxynol-9), and cationic surfactants (e.g. trimethyltetradecylammonium bromide, benzalkonium chloride, benzethonium chloride, myristyl gamma picolinium chloride) may be used. Thickeners, such as gelatin, natural gums and cellulose derivatives (such as those listed above as suspending agents) may be added. Buffers, such as citrate and phosphate buffering agents, may be included, as well as osmotic agents, such as sodium chloride and mannitol. For pharmaceutical compositions, which are to be administered orally, flavoring agents, sweeteners (e.g., mannitol, sucrose, sorbitol and dextrose), colorants and fragrances may be employed. In pharmaceutical compositions, excipients such as sorbitan monooleate (available as Span 80® from Sigma-Aldrich) and phosphatidylcholine (available as Phospholipon 90H from American Lecithin Company) may be employed.

Before the non-aqueous liquid composition is brought into contact with the dissolution medium for the dissolution assay, a non-aqueous liquid diluent is added to the non-aqueous liquid composition to obtain a diluted non-aqueous liquid composition. The non-aqueous liquid diluent is typically liquid at the contacting temperature and may be miscible, partially immiscible, or immiscible with water. The non-aqueous diluent can be selected from the same group of compounds, which were mentioned above with respect to the non-aqueous base, and can be the same or different as the non-aqueous base. Additionally, the non-aqueous diluent may contain an organic solvent. The diluent may also contain surfactants to affect the interfacial tension between the sample and the drug release medium.

The non-aqueous diluent may have a density greater or less than the drug release medium, but when the diiuent is combined with the sample, the combined composition will have a density less than that of the drug release medium. The non-aqueous diluent should not react in a deleterious manner with any of the components of the non-aqueous liquid composition or the aqueous dissolution medium. The non-aqueous diluent is preferably selected from the group consisting of natural oils, synthetic oils, and organic solvents. The non-aqueous diluent may also consist of or contain silicone-type oils (e.g. polydimethylsiloxane and polymethylhydrogensiloxane). The organic solvent can be selected from the group consisting of alcohols, aliphatic hydrocarbons, aromatic hydrocarbons, chlorinated hydrocarbons, glycols, glycol ethers, esters, ethers, ketones, petrochemicals, turpentine, dimethylformamide, and mineral spirits. More preferably the non-aqueous diluent is a natural or synthetic oil.

Illustrative examples of natural oils are canola oil, coconut oil, corn oil, peanut oil, sesame oil, olive oil, palm oil, safflower oil, soybean oil, cottonseed oil, rapeseed oil, sunflower oil and mixtures thereof. Of these, coconut oil and cottonseed oil are preferred and coconut oil is particularly preferred. The non-aqueous diluent may be modified by through peroxidation or other means known in the art as described above for the non-aqueous base.

A surfactant can also be added to the non-aqueous diluent in order to manipulate the surface free energy of the non-aqueous phase and the interfacial tension between the non-aqueous layer and the aqueous dissolution medium. Typical useful surfactants are non-ionic, cationic, anionic and zwitterionic surfactants. Illustrative examples of surfactants suitable for use in the present invention are sodium dodecyl sulfate, polyoxyethylene sorbitan monoleate (Tween 80™), chenodeoxycholic acid, glycocholic acid sodium salt, poly(oxytheylene)ₙ-sorbitan- monolaurate (Tween 20™), Taurocholic acid, octylphenol ethylene oxide condensate (Triton 100™), and hexadecyltrimethylammonium bromide, and polysiloxanes.

The type and amount of the surfactant will depend on the specific system of analyte, non-aqueous liquid composition and aqueous dissolution medium and can be determined by a person skilled in the art. Surfactant concentrations may be above or below the critical micelle concentration. Typical concentration ranges for the surfactant are from about 0.001% to about 1%.

In a preferred embodiment the non-aqueous diluent is a natural oil, optionally an oxidized natural oil.

The amount of the non-aqueous diluent tha is added to the non-aqueous liquid composition is not particularly limiting but is such that it improves the spreading behavior of the composition non-aqueous liquid. The ratio of the the non-aqueous diluent to the non-aqueous liquid composition typically ranges from 1:20 to 20:1, by volume, but can be much lower or higher. The exact amount may vary depending upon the nature of the analyte, non-aqueous base, and the dissolution medium. The appropriate amount of the composition and amount of non-aqueous diluent may be determined by one skilled in the art by iterative empirical evaluations. The relative amount of the composition and diluent may be considered to be optimal when the diluted composition spreads evenly across the surface of the drug release medium, or when adequate precision of repeat measurements is obtained.

Without wishing to be bound by this theory, it is assumed that the addition of the non-aqueous diluent, modifies and normalizes the spreading behavior of the non-aqueous liquid composition upon the surface of the aqueous dissolution medium in the dissolution testing apparatus. Without the addition of the non-aqueous diluent, even if the same non-aqueous liquid composition is employed in repeat measurements, it has been observed that the non-aqueous liquid composition can spread to different extents on the aqueous dissolution medium. This is believed to result in variations in the size of the contact area between the non-aqueous liquid composition and the aqueous dissolution medium. As a consequence, the dissolution rate of the analyte into the aqueous dissolution medium is affected by variable contact surface area and the obtained results can be imprecise and unreliable. When the non-aqueous diluent is added, the diluted non-aqueous liquid composition tends to spread to approximately the same extent not only if the same sample is repeatedly applied to the surface of aqueous dissolution media but also if different samples of the similar non-aqueous liquid compositions are investigated. Therefore, the size of the contact area between the non-aqueous liquid composition and the aqueous dissolution medium remains essentially the same and the precision and reliability of the results are improved.

After addition of the non-aqueous diluent to the non-aqueous liquid composition and mixing, at least part of the resultant diluted non-aqueous liquid composition and an aqueous dissolution medium are introduced into a dissolution testing apparatus. The order of adding the diluted non-aqueous liquid composition and the aqueous dissolution medium is not restricted. They can be added simultaneously or consecutively. In general the aqueous dissolution medium will be introduced into the dissolution testing apparatus first and the diluted non-aqueous liquid composition will be subsequently added.

Dissolution testing apparatuses are well-known in the analytical art and some have been standardized e.g. in various pharmacopeia such as the United States Pharmacopeia or the Japanese Pharmacopeia. Illustrative examples of dissolution testing apparatus are the rotating basket method (e.g. USP 1), the paddle method (e.g. USP II), various flow through methods (e.g. USP IV), the reciprocating cylinder apparatus (e.g. USP III) and various transdermanl dissolution testing apparatus (e.g Franz diffusion cell). The measurement of drug release from liquid samples and especially non-aqueous liquid dosage forms is often difficult, and standardized techniques for liquid samples have not been adopted.

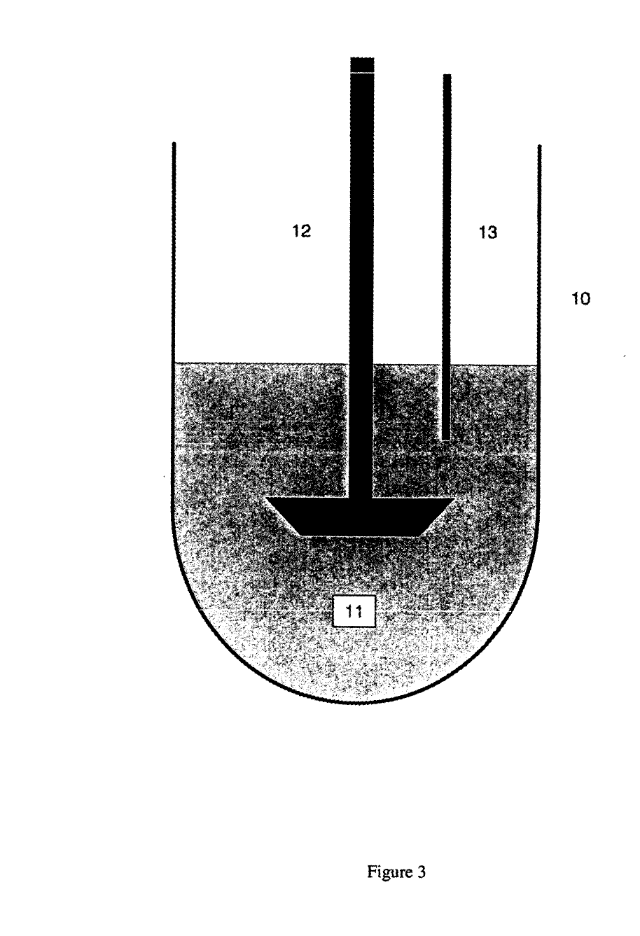

In one embodiment of the invention a paddle assembly is employed as the dissolution testing apparatus. A typical paddle assembly is illustrated in FIG. 3. It comprises a vessel 10, which contains the aqueous dissolution medium 11. In the methods of the present invention the diluted non-aqueous liquid composition is typically applied onto the surface of the aqueous dissolution medium, e.g. using a syringe or a pipette. The diluted non-aqueous liquid composition and the aqueous dissolution medium are stirred using the paddle 12. Samples of the aqueous dissolution medium can either be taken, e.g. by using a syringe or by employing a permanent sampling tube 13, which is optionally present in the paddle assembly. These types of dissolution apparatus are available commercially from a number of sources e.g. VanKel (Varian Inc.), Distek Inc., and Hanson Research Corporation.

The aqueous dissolution medium can be any aqueous dissolution medium known in the art. Commonly used dissolution media are water, hydrochloric acid (e.g. having a concentration in the range of from about 0.001 molar to about 0.1 molar HC1), simulated gastric fluid with or without pepsin, various buffer solutions (glycine, citrate, acetate, phosphate, and borate buffers), simulated intestinal fluids with or without enzymes (e.g. 0.05 molar phosphate buffer at pH 7.5 with or without pancreatin), water containing a surfactant, buffer solutions containing a surfactant, and aqueous alcoholic solutions (e.g. low molecular weight alcohols soluble in water typically containing 5 or less carbons to act as a cosolvent). These various parameters may be adjusted to alter solubility conditions for a given analyte. Through iterative experimentation, it is possible to empirically derive an optimal composition for a drug release medium, which may allow the experimenter to adjust the in vitro drug release rate to within a desired range. Adjustments in the solubility conditions may also allow the experimenter to discriminate in vitro between lots which behave differently in vivo.

In a preferred embodiment of the present invention a buffer solution, optionally containing a surfactant, is employed as the aqueous dissolution medium. The type of buffer solution is not particilarly restricted but should be selected depending on the specific system to be characterized. Buffer solutions may be selected to control the solubility of the analyte in the drug release medium, optimize the drug release profile, and optimize the degree of discrimination between important samples. Illustrative examples of buffer solutions are 0.05 molar glycine buffer at pH ranging from 2 to 3, 0.05 molar citrate buffer at pH 3, 0.05 molar acetate buffer at pH ranging from 4 to 5, 0.05 molar acetate buffer in normal saline at pH 5.5, 0.05 molar phosphate buffer at pH ranging from 6 to 8, potassium free 0.05 molar phosphate buffer at pH 6.8, 0.05 molar phosphate buffer in normal saline at pH 7.4, 0.05 molar borate buffer at pH ranging from 8 to 10). Preferred buffer solutions are 0.05 molar phosphate buffers with pH ranging from 6-7. The buffer can have any suitable molarity, for example from about 0.001 M to about 0.5 M, preferably from about 0.01 to about 0.1. However, it has been found that the precision and reliability of the methods of the invention can be further increased by employing a buffer having a low molarity. Therefore, in one embodiment of the invention, the molarity of the buffer is in the range of from about 0.1 to about 10 mM, more preferably from about 0.5 to about 2 mM. The selection of a low molarity buffer improves the spreading behavior of the non-aqueous liquid composition upon the surface of the drug release medium, and reduces unwanted interactions between the non-aqueous liquid composition and components of the drug release apparatus (e.g. agitation shaft). By improving the uniformity of spreading and minimizing unwanted physical interactions, it is possible to improve the precision and reliability of the analytical method. Information on dissolution buffer preparation can also be found in USP 24, pp. 2231-2240, United States Pharmacopeial Convention Inc, Jan. 1, 2000.

In a further preferred embodiment the aqueous dissolution medium is water, optionally containing a surfactant.

Optionally, the aqueous dissolution medium can contain a surfactant, which is another way to manipulate the solubility of the system. Typical useful surfactants are non-ionic, cationic, anionic and zwitterionic surfactants. Illustrative examples of surfactants suitable for use in the present invention are sodium dodecyl sulfate, polyoxyethylene sorbitan monoleate (Tween 80™), chenodeoxycholic acid, glycocholic acid sodium salt, poly(oxytheylene)ₙ-sorbitan- monolaurate (Tween 20™), Taurocholic acid, octylphenol ethylene oxide condensate (Triton X-100™), and hexadecyltrimethylammonium bromide.

The type and amount of the surfactant will depend on the specific system of analyte, non-aqueous liquid composition and aqueous dissolution medium and can be determined by a person skilled in the art. Surfactant concentrations may be above or below the critical micelle concentration. Typical concentration ranges for the surfactant are from about 0.001% to about 1%.

The pH of the aqueous dissolution medium should be selected depending on the specific system investigated. Generally the pH of the aqueous dissolution medium will be in the range from about 1 to about 10, preferably from about 2 to about 8. It is commonly known that the pH of the aqueous dissolution medium may affect the solubility of the analyte, and is one method of manipulating the sink conditions in the experiment. By optimizing the pH of the aqueous dissolution medium, it is possible to manipulate the dissolution characteristics of some analytes. In the case of pharmaceuticals, this may make it feasible to develop a correlation between the in vitro drug release characteristics and the in vivo pharmacokinetic performance.

As an aqueous dissolution medium, a particularly preferred system is an aqueous buffer having an odtimal pH value.

Aqueous dissolution media employed in the methods of the present invention can be prepared using any type of water such as deionized water, double distilled water or high purity water (i.e. having a resistance of at least about 1 megaohm, more preferably having a resistance of at least about 18 megaohms). Although it is not preferred, tap water can also be used as long as the constituents do not interfere with the measurement. Preferably double distilled water or high purity water, more preferably high purity water, are employed. The use of purer water, especially in combination with a low molarity buffer, has also been observed to increase the precision and reliability of the test results. High purity water can e.g. be provided by using a water purification apparatus such as the Milli-Q water purification systems available from Millipore Corporation (Bedford, Mass.). Typically the resultant high purity water has a resistance of about 18 M□. The selection of high purity water improves the spreading behavior of the non-aqueous liquid composition upon the surface of the drug release medium, and reduces unwanted interactions between the non-aqueous liquid composition and components of the drug release apparatus (e.g. agitation shaft). By improving the uniformity of spreading and minimizing unwanted physical interactions, it is possible to improve the precision and reliability of the analytical method.

The amount of non-aqueous liquid composition which is introduced in the dissolution testing apparatus can vary widely depending upon various factors, such as the nature of the dosage form (e.g. concentration of active ingredient, unit dose), the volume of the dissolution medium, the size of the contacting surface of the composition with the disoluiton medium. Typically the ratio of diluted non-aqueous liquid composition to aqueous dissolution medium is from about 1:20 to about 1:500 (v:v). In one embodiment of the invention it has been observed that the correlation of in vitro drug release with in vivo pharmacokinetic performance could be reversed (i.e. a negative correlation could become a positive correlation) by only introducing small amounts of non-aqueous liquid composition into the dissolution testing apparatus. In this case the ratio of non-aqueous liquid composition to aqueous dissolution medium is from about 1:2,000 to about 1:100,000 (v:v), preferably from about 1:20,000 to about 1:40,000.

When the diluted non-aqueous liquid composition has been introduced into the dissolution testing apparatus, the diluted non-aqueous liquid composition and the aqueous dissolution medium are contacted for a predetermined time. To improve contact between the diluted non-aqueous liquid composition and the aqueous dissolution medium, they are usually agitated, e.g. by stirring. The duration of contact can vary greatly and will depend, for example on the amount of agitation, the analyte, the non-aqueous liquid composition, the dissolution medium, the temperature, the sensitivity of the detection method used to determine the amount of analyte and a number of other factors. Furthermore, the duration of contact will depend on whether information on short term, medium term or long term dissolution rates or a combination of these is desired. Generally the duration of contact is from 5 minutes up to 24 hours, preferably until about 90% of the total amount of analyte has been dissolved. Typically the contacting will be conducted for from about 15 minutes to about 120 minutes, preferably from about 15 minutes to about 60 minutes.

During the contacting step, the aqueous dissolution medium can be held at any desired contacting temperature. Commonly the dissolution medium is held at a constant temperature of about 37° C. However, higher temperatures can be used to increase and lower temperatures can be employed to slow the dissolution rate. Since the temperature of the dissolution medium influences the dissolution rate if the results of more than one experiment are to be compared, the same temperature should be chosen for each experiment. Within the context of the invention the “same temperature” means that the differences between the temperatures of different experiments are at most 5° C., preferably at most 2° C. Preferably the contacting temperature is 37° C.

The amount of agitation during contacting such as the stirring rate also influences the dissolution rate of the analyte and the optimal conditions should be determined based e.g. on the size and shape of the paddle (if present), the geometry of the dissolution testing apparatus, and the amount and viscosity of the dissolution medium. Optimal conditions for stirring may be determined through iterative experimentation by one skilled in the art. Typically, optimal stirring conditions result in a surface that is smooth (no visible splashing or standing wave patterns), from the outer edge of the vessel to the center, including the area in which the agitation shaft contacts the dissolution medium (i.e. the surface does not exhibit a vortex “cone” caused by the surface of the drug release medium being distorted downward by mixing). Typically the stirring speed will be in the range from about 25 to about 100 rpm, preferably from about 50 to about 75 rpm.

In the prior art a wide variety of modifications of the standardized dissolution testing apparatus such as paddle assemblies have been suggested. In the methods of the present invention the standardized dissolution testing apparatuses known in the art as the USP I and USP II apparatuses can be reliably employed without any modifications.

After the predetermined amount of time the amount of analyte in the aqueous dissolution medium is determined. With some detection methods the amount of analyte can be determined while the aqueous dissolution medium remains in the dissolution testing apparatus, typically, however, at least part of the aqueous dissolution medium is removed from the dissolution testing apparatus, e.g. by means of a syringe or the sampling tube 13. Although it is possible to use all of the aqueous dissolution medium for the analysis and this might be necessary with some detection methods, generally only part of the aqueous dissolution medium will be employed. The size of the sample removed for determining the amount of analyte will depend on a variety of factors, particularly on the employed detection method, and can e.g. be from about 0.1 to about 100 mL, preferably from about 1 to about 20 mL.

If desired, the sample of the aqueous dissolution medium, which is to be used for determining the amount of analyte, can be filtered after it has been removed from the dissolution testing apparatus. This removes particles of foreign matter and undissolved analyte which might interfere with the determination of the dissolved analyte and confound the measurement. Filtration can be achieved by any suitable means such as filtering through a filter having an average pore size of from about 0.1 to about 50 microns, preferably from about 0.1 to about 0.5 microns. These filters are, for example commercially available under the trade designations Acrodisk® from Gelman Laboratory.

After the optional filtering step the amount of analyte in the aqueous dissolution medium is determined. Any analytical method suitable for determining the amount of analyte can be employed. The choice of the analytical method will depend on a variety of parameters including the nature of the analyte, its concentration range, the dissolution medium, and also which methods are available in the laboratory. Illustrative examples of analytical methods are separation techniques (e.g. high performance liquid chromatography, liquid chromatography, thin layer chromatography, capillary electrophoresis, gas chromatography), photometric and spectrophotometric techniques (e.g. ultraviolet-visible (UV-Vis), Fourier transform infrared (FTIR), atomic absorption (AA), atomic emission (AE), mass spectrometry (MS)). Chromatographic methods, in particular gas chromatography (GC) and high performance liquid chromatography (HPLC), are preferred. Examples of suitable chromatographic methods are reverse phase high performance liquid chromatography (RP-HPLC) and normal phase high performance liquid chromatography (NP-HPLC), incorporating any of a variety of detection techniques known in the art. Examples of detection techniques which may be used in conjunction with a suitable chromatographic method include, UV-Vis, index of refraction, mass spectrometry and light scattering detection. Flow injection analysis (FIA) with UV-Vis detection can also be employed as an analytical method. FIA is particularly suitable when a high throughput of samples is needed, such as is the case when performing in-process characterization of a manufacturing system in real time

The methods of the invention has been explained supra with respect to an embodiment in which the amount of analyte dissolved at a single predetermined point of time is determined. In many cases, it is of interest to monitor the dissolution rate over a period of time to determine whether the analyte is released at a constant rate or if the rate varies with time (e.g. a large amount at the beginning of the dissolution testing and then lesser amounts later on). In these cases, it is possible to use a sufficiently large dissolution testing apparatus, to remove two or more samples therefrom at different predetermined times and to analyze these samples individually. It is also possible to prepare two or more identical experiments and to contact them under identical conditions with the exception that the duration of agitation is varied. The aqueous dissolution medium sampled at the various points of time from these separate experiments is analyzed individually. The results can then be used to determine the time-dependent profile of the dissolution rate.

Using the methods of the invention, it is now possible to reliably and accurately measure the dissolution rate of an analyte in a non-aqueous liquid composition. A significant reduction in the variability of results of repeat measurements is observed. In pharmaceutical applications, the methods of the invention make it possible to develop a useful correlation between the in vitro methods of the invention and in vivo pharmacokinetic studies. Therefore, they can be used as a rugged and reliable method in quality control during the manufacture of pharmaceuticals to ensure adequate bioperformance and lot consistency. Since the methods are simple, cheap and fast, and can be conducted with a standardized dissolution testing apparatus, they can also be used with advantage in the development of pharmaceuticals and their dosage forms.

### EXAMPLES

The following examples are presented to illustrate the invention. However, they should not be construed as limiting.

Precision:

The precision of the methods of the present invention can be determined by calculating the relative standard deviation (RSD) of repeat measurements. Typically, the relative standard deviation is determined by measuring the dissolution rate of an analyte under identical conditions with replication greater than two. The relative standard deviation is then calculated according to the following formula:

$$RSD = \frac{s . d .}{\overline{X}} \times 100$$

where s.d. is the standard deviation which is defined as:

$$s . d . = \sqrt{\frac{\sum {( X - \overline{X} )}^{2}}{N - 1}} ;$$

X is the individual result; N is the number of replicants; and \overline{X}0 is the mean.

Preferably the relative standard deviation is 10% or less, more preferably 2% or less.

Accuracy:

The accuracy of the methods of the present invention can be determined by measuring the transfer of an analyte from a non-aqueous liquid composition to an aqueous medium where the non-aqueous liquid composition is spiked with a known amount of analyte. The spiked non-aqueous liquid composition is equilibrated with the aqueous drug release medium by shaking or stirring, after which the amount of analyte in the aqueous dissolution medium is determined. The concentration of analyte which transferred to the aqueous medium is then compared to the concentration which would result, in theory, if 100% of the analyte had transferred. (e.g. under the assumptions that no pipetting, weighing errors or losses occur, that 100% of the analyte has dissolved and that 100% of the analyte is detected). Methods of the invention are accurate within the range of from about 70% to about 100%, preferably from about 90% to about 100%.

General Dissolution Procedure

Unless otherwise mentioned the following general procedure was followed.

Dissolution Conditions:

Apparatus: USP II (rotating paddle), with covered vessels. Lock sampling probes into place half the distance between the surface of the medium and the paddle. Install luer-lock adapters on the sampling probe tubing to facilitate removal of samples from the apparatus. All samples must be removed via these adapters. The dissolution flasks and paddles must be thoroughly cleaned. (See DRA Cleaning Procedure.) Residues from soap or alcohol may affect results.

| | |
|---|---|
| Flask Size: | 1000 mL |
| Dissolution | 500 mL of 0.001 M pH 7.0 phosphate at 37° C. ± 0.5° C. |
| Fluid: | |
| Stock | Dissolve 3.9 g of potassium phosphate monobasic |
| Buffer: | (KH₂PO₄) and 3.7 g of potassium phosphate dibasic |
| | (K₂HPO₄) in Milli-Q water, or equivalent in a |
| | one liter volumetric flask. Dilute to volume with |
| | Milli-Q water, or equivalent and mix. Check the |
| | pH by dilution 10 mL of the Stock Solution to 500 mL |
| | with Milli-Q water. The pH should be 7.0 ± 0.1. |
| | If necessary, adjust the pH of the Stock Solution |
| | with 50% sodium hydroxide or concentrated |
| | hydrochloric acid. Recheck that the pH of the |
| | Working Solution is 7.0 ± 0.1. |
| Working | Dilute 10 mL of Stock Solution to 500 mL with |
| Buffer: | Milli-Q water. Degas before use. |
| Stirring | 50 rpm |
| Speed: | |
| Sample | 10 mL |
| Volume: | |
| Filter: | Acrodisc (Gelman) 0.2 micrometer disposable (no. 4496), |
| | or equivalent |

Working Standard Preparation:

Accurately weigh out approximately 1 mg of Ceftiofur Hydrochloride Reference Standard into a 100 mL volumetric flask. Wet the drug with approximately 1 mL of methanol to dissolve (sonicate if necessary). Dilute to volume with Working Buffer. Prepare at least 2 working standard solutions.

Pharmaceutical Non-Aqueous Sample Preparation: Re-suspend each bottle of Ceftiofur Crystalline Free Acid (CCFA) Suspension, the preparation of which is described herein below, until there is no visible sign of drug on the bottom of the vial. Dilute the sample 1:1 (v/v) with hydrogenated coconut oil (available as Miglyol 812, from HulsAmerica) prior to dissolution assay as follows: Using a calibrated positive displacement pipette, add equal volumes of CCFA Suspension and Miglyol to a suitable container (e.g., 20 mL screw cap vial). The actual volume used is not critical, as long as the dilution is precisely 1:1. Suggested volumes range from 1.0 mL to 5.0 mL for each component.

Mix the diluted sample thoroughly by hand and by vortex mixer, then withdraw 50 microliters into a calibrated positive displacement pipetman. Wipe excess suspension from tip and dispense the contents drop wise onto the surface of the medium in each dissolution flask under agitation. Apply the drops so that the tip of the pipetman is about ½ inch from the surface of the medium, and about ½ way between the side of the vessel and the sample probe. Dip the tip of the pipetman into the media to remove the remaining traces of suspension. Stagger the sample application to each subsequent flask to allow for sampling time. All samples should be dispensed into the dissolution flasks as soon after dilution as possible.

At the time(s) specified (e.g. 15, 30, 60, and 120 minutes) withdraw 10 mL of dissolution fluid (a 10 mL disposable syringe works well) and filter with an Acrodisk part number 4496. Discard the first 5 mL of filtrate, and then collect an appropriate volume of filtrate in an HPLC autosampler vial. Stagger the sample removal process in the same manner as used for sample application. Proceed to quantitative HPLC analysis.

Chromatographic Conditions:

Equipment:

| | |
|---|---|
| HPLC Pump: | A suitable pump capable of isocratic operation |
| | at 3000 psi (e.g. Agilent 1100 from Agilent |
| | Technologies). |
| Injector: | A suitable low dead volume injector |
| Detector: | 254 nm |
| Column: | Waters Symmetry C8 3.9 × 50 mm, 5 micron, |
| | or equivalent |
| Injection Volume: | about 20 mcl |

Chromatographic Operating Parameters:

| | |
|---|---|
| Attenuation: | Adjust as appropriate |
| Chart Speed: | Adjust as appropriate |
| Flow Rate: | Approximately 1.0 mL/min (may be adjusted). |
| Pressure: | Approximately 2000 psi |

HPLC Mobile Phase: For 1 liter of Mobile Phase:

Add 3.85 g ammonium acetate and 13.5 mL of 40% tetrabutylammonium hydroxide to an appropriate container. Dilute to 700 mL with Milli-Q or HPLC grade water. Adjust the pH to 6.7±0.1 with glacial acetic acid. Filter the aqueous buffer through a 0.45 micrometer membrane filter. To the 700 mL of aqueous buffer add 200 mL of methanol and 110 mL THF and mix. Sonicate under vacuum to degas.

Ouantitative HPLC Analysis:

Analyze filtered samples by HPLC. Suitable reference standards solutions should be placed at the beginning and end of each chromatographic run with not less than six standard injections per run. Bracket each set of six samples with reference standard solutions. Suitable blank solutions should be analyzed periodically to monitor injections system for potential carryover.

System Suitability Test:

The Relative Standard Deviation of the Standard Factor should not be more than 2.0%.

The Standard Factor (SF) may be calculated from the following formula:

SF═P×(Wstd/Rstd)

where,

P=Purity of Reference Standard, expressed as percent

Wstd=Weight of Reference Standard

Rstd=Standard peak area

Calculations:

Calculate percent ceftiofur released at each time point using the following equation correcting for volume removed:

$$\% Dn = ( \frac{Rsam}{Rstd} ) \times ( \frac{C s}{L} ) \times ( \frac{p}{Vsus} ) \times ( V - ( ( n - 1 ) \times S V ) ) + ( ( D1 + D2 + … D n - 1 ) \times \frac{S V}{V} )$$

where,

Dn=Percent dissolved at nth test point

Rsam=Sample peak area

Rstd=Standard peak area

Cs=Concentration of Working Standard, in mg/mL

L=Label strength of CCFA Suspension. (200 mg/mL)

P=Purity of Reference Standard, expressed as percent

Vsus=Volume of CCFA Suspension applied is 0.025 mL (since 50 mcl of 1:1 dilution was applied)

V=Initial volume of Dissolution Fluid, in mL

n=Test point number

SV=Sampling volume, in mL

D1=Percent dissolved at first test point

D2=Percent dissolved at second test point

Dn-1=Percent dissolved at the (n-1) test point

DRA Cleaning Procedure:

Saturate Kimwipes with 3A alcohol and wipe paddles thoroughly to remove residue. Allow to air dry. Dispose of the aqueous buffer containing samples of non-aqueous composition. Rinse the vessel with 3A alcohol and clean most of the residue on the flask by wiping with Kimwipes. Rinse with 3A alcohol and place vessel back in the DRA.

Using a glass syringe, inject about 10 mL of Dimethylformamide (DMF) through sampling line from the sample manifold, collecting the waste in the drug release vessel. Follow with 10 mL of 3A alcohol. Remove the vessel, and use Kimwipes to absorb the solvent mixture and clean the inside surface of the vessel. Follow with a 3A alcohol rinse and dry.

Flush the lines with deionized water, and then blow air through the lines with an empty syringe. If solvents splash on paddles during cleaning of lines, repeat paddle cleaning procedure.

Test Materials

The following procedures were employed to produce the experimental pharmaceutical non-aqueous suspensions used in the examples cited below.

Ceftiofur Crystalline Free Acid (CCFA) Suspension 100 mg/mL in Cottonseed Oil:

Lots 40,620 and 40,700 were prepared following the same manufacturing process. The nonaqueous vehicle was prepared by pumping cottonseed oil into a jacketed vessel and heating to 115° C. Phospholipon 90H was added (0.05% by weight) (available from American Lecithin Co.) and mixed. The solution was cooled to 45° C. Sorbitan monooleate (available as Span 80® from Sigma-Aldrich) was added (0.15% by weight) and mixed. CCFA was added at 100 mg/mL and mixed through a triblender until the suspension was homogeneous. The suspension was recirculated through the triblender, with tank agitator running and screened. The resultant suspension was filled in sterile vials, stoppered and oversealed. The sealed vials were sterilized using gamma irradiation. The lots were labeled 40,700 and 40,620.

Ceftiofur Crystalline Free Acid (CCFA) Suspension 200 mg/mL in Cottonseed with Miglyol Oil:

A substantially peroxidized unsaturated oil was prepared from natural cottonseed oil. 105 parts by volume of natural cottonseed oil were added to a vessel having a steam jacket for heating. Steam was applied to the jacket to heat the oil to between about 85 and about 100° C. Air was bubbled through the oil while it was agitated. The flow rate of the air varied from about 1 standard cubic foot per hour (SCFH)/liter to 20 SCFHI/liter.

Agitation was such that the temperature of the oil remained constant over the time period of heating. The oil was heated for a time and at a temperature necessary to achieve a peroxide value as measured by the method of the US Pharmacopeia (USP 24 NF 19 at page 1870) or by AOCS method 8-53 and then cooled, transferred to a different container and stored under nitrogen conditions. To achieve a peroxide value of about 10, at a temperature of about 89° C. the oil was heated for about 9 hours, at a temperature of about 100° C. the oil was heated for about 3 hours, and at a temperature of about 105° C. the oil was heated for about 2.3 hours. To achieve a peroxide value of about 40, at a temperature of about 100° C. the oil was heated for about 6.75 hours, and at a temperature of about 105° C. the oil was heated for about 5.5 hours. To achieve a peroxide value of about 80, at a temperature of about 105° C. the oil was heated for about 8 hours. The relationship between the time and temperature of the oil as compared to its peroxide value is considered to be linear and one skilled in the art could achieve a desired peroxide value depending on the time and temperatures selected for processing. The oxidized oil may be diluted with fresh oil to bring about the preferred end peroxide value.

Following preparation of the peroxidized unsaturated oil, the CCFA 200 mg/mL formulation was compounded as follows: 10 to 20 parts by volume of the peroxidized cottonseed oil having a peroxide value of between about 10-200 were mixed with 80 to 90 parts by volume of Miglyol 812 (available from HulsAmerica) to form a carrier vehicle. 0.2 parts by weight of CCFA were added and mixed for 1-3 hours to form a uniform suspension such that the concentration of CCFA was 200 mg/mL. The suspension was heated to about 80-110° C. for about 0.1 to 10 days and permitted to cool. The suspension was packaged and sterilized with gamma radiation if desired. Experimental parameters for each of the lots employed in the following examples are detailed in Table 1.

Ceftiofur Crystalline Free Acid (CCFA) Suspension 100 mg/TnL in Cottonseed with Miglyol Oil:

The procedure detailed above for the 200 mg/mL formulation is repeated except that the ratio of modified cottonseed oil to Miglyol 812 is 10:90, and in step the amount of CCFA added is such that the concentration of CCFA is 100 mg/ml.

TABLE 1
CCFA Suspension Manufacturing Parameters for Designated Lots.

| | | Nominal | | | |
|---|---|---|---|---|---|
| | | Peroxide | CCFA | | Heat: Time |
| | CSO:Miglyol | Value of | concentration | | and |
| Lot ID | Proportionality | CSO | (mg/mL) | Irradiated? | Temperature |
| SFH-134 | 20:80 | 100 | 200 | No | 5 hr at |
| | | (diluted | | | 100° C. |
| | | from PV258) | | | |
| SFH-135 | 10:90 | 200 | 200 | No | 20 hr at |
| | | | | | 100° C. |
| SFH-148-42 | 20:80 | 80 | 200 | No | 42 hr at |
| Hr | | | | | 100° C. |
| SFH-148-14 | 20:80 | 80 | 200 | No | 14 hr at |
| Hr | | | | | 100° C. |
| SFH-148-7 | 20:80 | 80 | 200 | No | 7 hr at |
| Hr | | | | | 100° C. |
| SFH-148-2 | 20:80 | 80 | 200 | No | 2 hr at |
| Hr | | | | | 100° C. |
| SFH-146 3.5 | 20:80 | 80 | 200 | No | 3.5 hr at |
| Hr | | (diluted | | | 100° C. |
| | | from PV258) | | | |
| SFH-10 | 20:80 | 80 | 0 | No | None |
| | | | (Placebo) | | |
| SFH-11 | 20:80 | 80 | 200 | No | 10 hr at |
| | | | | | 100° C. |
| SFH-11-IRR | 20:80 | 80 | 200 | Yes | 10 hr at |
| | | | | | 100° C. |
| 51338 | 20:80 | 80 | 200 | No | 80 min at |
| | | | | | 100° C. |
| 51338-IRR | 20:80 | 80 | 200 | Yes | 80 min at |
| | | | | | 100° C. |
| SFH-95 | 10:90 | 73 | 100 | No | Var days at |
| | | | | | 80° C. |

### Example 1

This example illustrates the variations in spreading behavior.

Spreading behavior is a way of describing the phenomenon which occurs when one liquid phase is placed upon the surface of another immiscible liquid phase. Upon contact, the liquid may form a tight lens-shaped pool upon the surface of the other liquid, or it may spread evenly across the surface. Intermediate and variable spreading may also occur. Spreading behavior may be defined in mathematical (e.g. surface thermodynamics) or qualitative terms.

To compare the spreading behavior of different lots of CCFA Suspension, one mL of each suspension was gently applied through an 18 gauge needle to separate containers (plastic petri dishes) containing 25 mL of drug release medium. The suspension samples were applied drop wise upon the surface of the drug release medium.

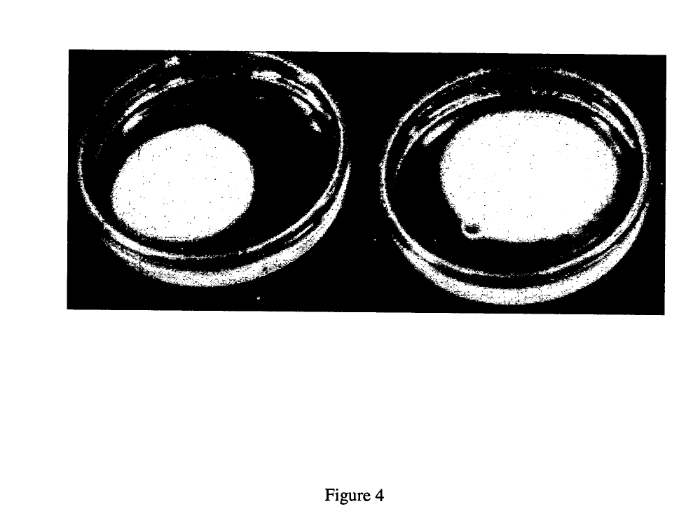

The spreading behavior was assessed by the size of the area of the pool of suspension upon the drug release medium after allowing for a sufficient length of time for a quasi equilibrium to be achieved (about 21 hours). A photograph of the suspension samples is shown in FIG. 4. The petri dish containing lot 40,700 is on left; the petri dish containing lot 40,620 is on the right. After 21 hours, the diameter of the pool of CCFA suspension upon the drug release medium was measured with a ruler. The diameter of the lens on lot 40,700 was 4.8 cm, while the diameter of the lens on lot 40,620 was 6.0 cm.

### Example 2

Example 2 shows the influence of diluting the non-aqueous liquid composition with an non-aqueous diluent.

The inconsistent spreading of an oil-based suspension upon the surface of the drug release medium, demonstrated in Example 1, was a significant obstacle to developing a useful USP II drug release assay for CCFA oil-based suspensions. Variable spreading behavior resulted in variable surface area of suspension in contact with drug release medium, thus affecting the drug dissolution rate. This in turn affected the quality of the correlation between in vitro drug release and in vivo pharmacokinetics.

The statistical significance of the correlation between in vitro drug release and in vivo pharmacokinetics was assessed as described below. Correlation is defined as the degree of association, or how well one variable can be predicted from another. One approach for assessing the degree of correlation between two variables is to statistically analyze the slope of the least squares fit. A significant correlation between variables occurs when the slope of the least squares fit is different from zero at 95% confidence (p≦0.05). If the slope is not different from zero at 95% confidence (p>0.05), the correlation is not significant.

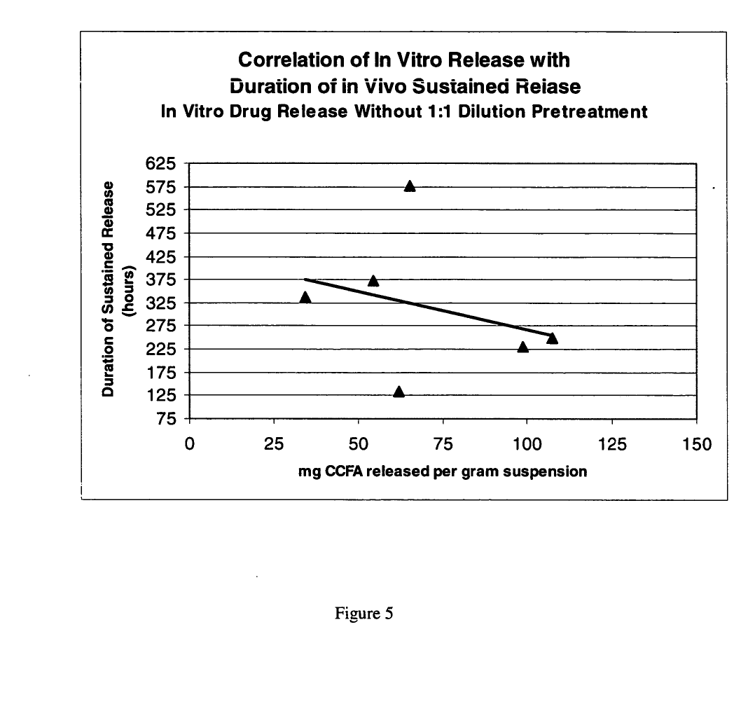

The impact of variable spreading behavior upon the correlation of in vitro drug release with in vivo pharmacokinetic performance can be seen in FIG. 5. In vitro drug release data for selected lots of CCFA are plotted versus their in vivo pharmacokinetic performance (i.e. duration of sustained release in hours). A least squares fit trend line is plotted as a solid line in FIG. 5. In this case, the in vitro drug release assay employed did not include diluting the non-aqueous suspension with an inert oil, and variable spreading behavior of the suspension lots was observed. A significant correlation was not observed between the in vitro drug release results and the duration of sustained release. The slope of the least squares fit was not significantly different from zero (p=0.57).

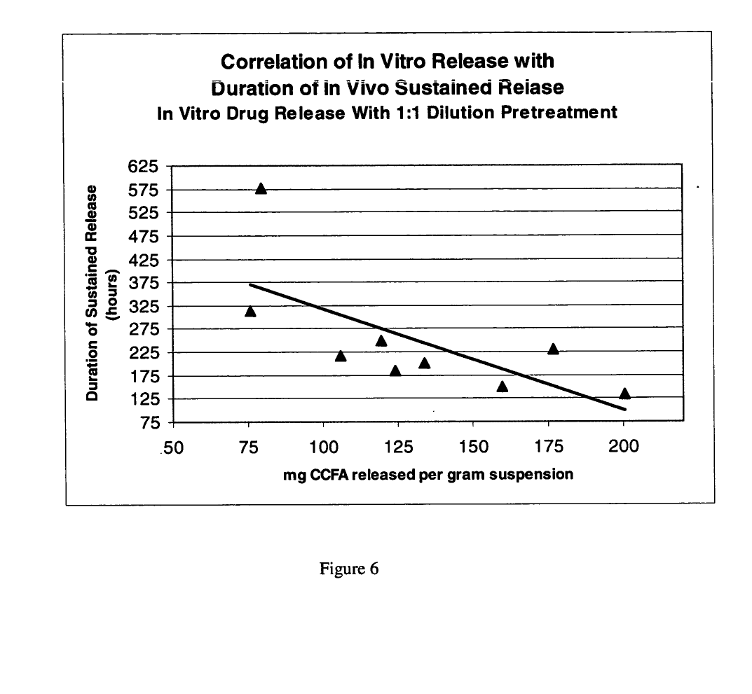

Diluting the non-aqueous suspension composition 1:1 with an inert oil resulted in a normalization of the spreading behavior. Incorporating the pre-dilution step into the invention resulted in the development of a useful in vitro/in vivo correlation (IVIVC). In vitro data on selected CCFA lots, obtained using the pre-dilution step, are plotted versus the duration of in vivo sustained release in FIG. 6, along with a least squares fit trend line. A significant correlation was observed between the in vitro drug release results and the duration of sustained release. The slope of the least squares fit was significantly different from zero (p=0.04).

### Example 3

Example 3 illustrates the effect of the ionic strength of the buffer on the precision of the measurement method.

During in vitro drug release testing of non-aqueous suspensions, the non-aqueous composition which “floats” upon the surface of the drug release medium, can interact with or adhere to the agitation shaft. The adherence or interaction of the sample with the shaft inhibits uniform spreading of the suspension upon the surface of the drug release medium. The extent and duration of the interaction is variable, which in turn, induces unwanted variability in assay results. Minimizing the ionic strength of the in vitro drug release medium reduces and may eliminate the interaction of the sample with the agitation shaft and enhances spreading. Dissolution buffers were prepared at 50 mM, 5 mM, and 1 mM. A single lot of CCFA (SFH-95) was assayed multiple times using each dissolution buffer. The assay variability, using the three dissolution buffers, was assessed by calculating the standard deviation of the results which are summarized in Table 2.

TABLE 2

standard deviation of analyte
concentrations sampled at

| ionic strength | 15 min | 30 min | 60 min | 120 min |
|---|---|---|---|---|
| 50 mM | 3.84 | 2.86 | 13.50 | 6.32 |
| 5 mM | 2.00 | 1.85 | 1.74 | 1.57 |
| 1 mM | 0.69 | 0.61 | 0.60 | 0.65 |

### Example 4

In this example the effect of the sample size is shown.

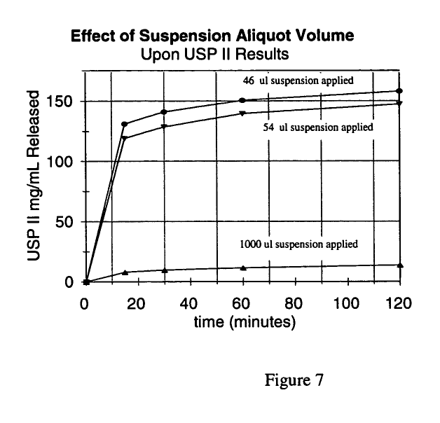

Drug release assays on CCFA Suspension lot SFH-11 were conducted as described in the General Dissolution Procedure above with the following modification: the volume of non-aqueous suspension applied to the surface of the drug release medium was varied from 46 to 1000 microliters. Results are summarized in FIG. 7. Reducing sample size increased the relative amount of drug dissolved during the test.

### Example 5

Example 5 illustrates the linearity of the HPLC quantitative analytical procedure employed in the method of the invention.

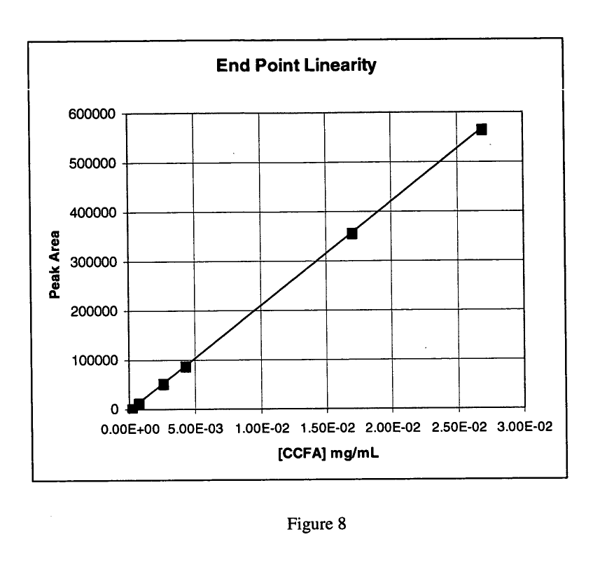

Six solutions of CCFA were prepared at concentrations ranging from 1.27×10⁴ to 2.68×10⁻² mg/mL. Aliquots from each solution were assayed and the peak areas were determined using the HPLC quantitative method and chromatographic parameters described above. Results are summarized FIG. 8.

### Example 6

Example 6 shows the recovery of an analyte from a non-aqueous liquid composition using the drug release media specified in the General Dissolution Procedure above.

The recovery of CCFA bulk drug dissolved in drug release media spiked with a 1:1 mixture of placebo lot SFH-10 and Miglyol 812 was assessed by spiking 15 microliters of 1:1 Placebo:Miglyol into 75 mL of standard solutions of CCFA. This spiking level (15 microliters in 75 mL) corresponded to 100 microliters of the Placebo:Miglyol mixture per 500 mL of aqueous medium. This represented a two fold increase in the relative concentration of non-aqueous phase to that specified in the General Dissolution Procedure, thus representing a “worst case” or conservative approach to assessing the potential for negative bias (i.e. incomplete recovery) in the assay. Recovery was determined at six concentrations of CCFA ranging from about 1 to 15 ppm of CCFA in the aqueous phase. For a 200 mg/mL CCFA product, these concentrations corresponded to about 10-150% dissolved. For example, the General Dissolution Procedure specifies measuring the drug release from 50 microliters (0.050 mL) of a 1:1 dilution of CCFA suspension in Miglyol 812 into 500 mL of drug release medium. If 10% of the drug dissolved, the resultant concentration of CCFA in the aqueous phase would be:

$$0.1 \times \frac{200 mg}{mL} \times \frac{0.050 mL}{2} \times \frac{1}{500 mL} = \frac{0.001 mg}{mL} ( or 1 ppm )$$

After spiking, the mixtures were equilibrated by shaking on a platform shaker at room two hours. The spiked samples were filtered and the concentration of the termined using HPLC procedure described in the General Dissolution e results are summarized in Table 3.

TABLE 3

| No. | mg/ml added | mg/ml measured | % recovery |
|---|---|---|---|
| 1 | 0.001008 | 0.00101767 | 100.96 |
| | | 0.00101058 | 100.26 |
| 2 | 0.002520 | 0.00253904 | 100.76 |
| | | 0.00252894 | 100.35 |
| 3 | 0.005040 | 0.00503141 | 99.83 |
| | | 0.00503060 | 99.81 |
| 4 | 0.007540 | 0.00755600 | 100.21 |
| | | 0.00755681 | 100.22 |
| 5 | 0.010082 | 0.01005470 | 99.73 |
| | | 0.01007000 | 99.88 |
| 6 | 0.014982 | 0.01497320 | 99.94 |
| | | 0.01495330 | 99.81 |

The average recovery of CCFA was 100.15%.

What is claimed is:

1. Method of determining the dissolution rate of an analyte in a non-aqueous liquid composition, comprising the steps of:
(a) providing a non-aqueous liquid composition comprising an analyte and a non-aqueous base;
(b) adding a non-aqueous diluent to the non-aqueous liquid composition to provide a diluted non-aqueous liquid composition;
(c) introducing at least part of the diluted non-aqueous liquid composition and an aqueous dissolution medium into a dissolution testing apparatus;
(d) contacting the diluted non-aqueous liquid composition and the aqueous dissolution medium for a predetermined time; and
(e) determining the amount of analyte in the aqueous dissolution medium.

2. The method of claim 1, wherein the amount of analyte in the aqueous dissolution medium is determined at several different predetermined times.

3. The method of claim 1, further including, in step (e), a step of filtering the aqueous dissolution medium, which is to be used for determining the amount of analyte in the aqueous dissolution medium, before determining the amount of analyte therein.

4. The method of claim 3, wherein the pore size of the filter ranges from about 0.1 to about 50 microns.

5. The method of claim 1, wherein the non-aqueous liquid composition is a pharmaceutical composition.

6. The method of claim 5, wherein the analyte is the pharmaceutically active component.

7. The method of claim 5, wherein the pharmaceutical composition is a sustained release dosage form.

8. The method of claim 5, wherein the pharmaceutical composition further contains pharmaceutically acceptable components selected from the group consisting of excipients, additives, suspending agents, preservatives, wetting agents, thickeners, buffers, flocculating agents, flavoring agents, sweeteners, colorants and fragrances.

9. The method of claim 1, wherein the analyte is selected from the group consisting of ACE inhibitor; α-adrenergic agonist; β-adrenergic agonist; α-adrenergic blocker; β-adrenergic blocker; alcohol deterrent; aldose reductase inhibitor; aldosterone antagonist; amino acid; anabolic; analgesic; anesthetic; anorexic; antacid; anthelmintic; antiacne agent; antiallergic; antiandrogen; antianginal agent; antianxiety agent; antiarrythmic; antiasthmatic; antibacterial agent; antialopecia and antibaldness agent; antiamebic; antibody; anticholinergic drug; anticoagulant; blood thinner; anticolitis drug; anticonvulsant; anticystitis drug; antidepressant; antidiabetic agent; antidiarrheal; antidiuretic; antidote; antiemetic; antiestrogen; antiflatulent; antifungal agent; antigen; antiglaucoma agent; antihistaminic; antihyperactive; antihyperlipoproteinemic; antihypertensive; antihyperthyroid agent; antihypotensive; antihypothyroid agent; anti-infective; anti-inflammatory agent; antimalarial agent; antimigraine agent; antineoplastic; antiobesity agent; antiparkinsonian agent; antidyskinetics; antipneumonia agent; antiprotozoal agent; antipruritic; antipsoriatic; antipsychotic; antipyretic; antirheumatic; antisecretory agent; anti-shock agent; antispasmodic; antithrombotic; antitumor agent; antitussive; antiulcerative; antiviral agent; anxiolytic; bactericidin; bone densifier; bronchodllator; calcium channel blocker; carbonic anhydrase inhibitor; cardiotonic; heart stimulant; chemotherapeutic; choleretic; cholinergic; CNS stimulant; coagulant; contraceptive; cystic fibrosis drug; decongestant; diuretic; dopamine receptor agonist; dopamine receptor antagonist; enzyme; estrogen; expectorant; glucocorticoid; hemostatics; HMG CoA reductase inhibitor; hypnotic; immunomodulator; immunosuppressant; laxative; miotic; monoamine oxidase inhibitor; mucolytic; muscle relaxant; mydriatic; narcotic antagonist; NMDA receptor antagonist; oligonucleotide; ophthalmic drug; oxytocic; peptide; proteins; polysaccharide; progestogen; prostaglandin; protease inhibitor; respiratory stimulant; sedative; serotonin uptake inhibitor; sex hormone; smoking cessation drug; smooth muscle relaxant; smooth muscle stimulant; thrombolytic; tranquilizer; urinary acidifier; vasodilators; and vasoprotectant.

10. The method of claim 1, wherein the analyte is a cephalosporin selected from the group consisting of ceftiofur, cefepime, cefixime, cefoperazone, cefotaxime, cefpodoxime, ceftazidime, ceftizoxime, ceftriaxone, moxalactam, pharmaceutically acceptable salts and derivatives thereof.

11. The method of claim 10, wherein the analyte is ceftiofur, a pharmaceutically acceptable salt or derivative thereof.

12. The method of claim 1, wherein the non-aqueous base is selected from a fat or wax.

13. The method of claim 12, wherein the non-aqueous base is a fat that is an oil.

14. The method of claim 13, wherein the oil is selected from the group consisting of canola oil, coconut oil, corn oil, peanut oil, sesame oil, olive oil, palm oil, safflower oil, soybean oil, cottonseed oil, rapeseed oil, sunflower oil and mixtures thereof.

15. The method of claim 12, wherein the oil is cottonseed oil.

16. The method of claim 1, wherein the non-aqueous liquid composition is a suspension, solution or emulsion.

17. The method of claim 1, wherein the non-aqueous liquid composition is a suspension.

18. The method of claim 1, wherein the non-aqueous diluent is selected from the group consisting of oils and organic solvents.

19. The method of claim 18, wherein the non-aqueous diluent is an oil.

20. The method of claim 19, wherein the oil is coconut oil or cottonseed oil.

21. The method of claim 1, wherein the amount of non-aqueous diluent is from about 0.25 to about 10 parts by weight relative to the amount of non-aqueous liquid composition.

22. The method of claim 1, wherein the contacting is conducted for a predetermined time to dissolve from about 10% to about 100% of the total amount of analyte, which was initially present in the non-aqueous liquid composition, in the aqueous dissolution medium.

23. The method of claim 22, wherein the stirring is conducted for a predetermined time to dissolve from about 10% to about 100% of the total amount of analyte, which was initially present in the non-aqueous liquid composition, in the aqueous dissolution medium.

24. The method of claim 1, wherein the aqueous dissolution medium is prepared using high purity water.

25. The method of claim 1, wherein the aqueous dissolution medium is selected from a group consisting of water, hydrochloric acid solution, simulated gastric fluid, buffer solution, simulated intestinal fluid, water containing a surfactant, buffer solution containing a surfactant, and aqueous alcoholic solution.

26. The method of claim 25, wherein the aqueous dissolution medium is a buffer solution.

27. The method of claim 26, wherein the buffer solution is selected from the group consisting of glycine buffer at pH ranging from 2 to 3, citrate buffer at pH 3, acetate buffer at pH ranging from 4 to 5, acetate buffer in normal saline at pH 5.5, phosphate buffer at pH ranging from 6 to 8, potassium free phosphate buffer at pH 6.8, phosphate buffer in normal saline at pH 7.4, and borate buffer at pH ranging from 8 to 10.

28. The method of claim 27, wherein the buffer solution has a molarity of from about 1 mM to about 10 mM.

29. The method of claim 27, wherein the buffer has a molarity of from about 1 mM to about 5 mM.

30. The method of claim 1, wherein in step (d) the ratio of non-aqueous liquid composition to aqueous dissolution medium is from about 1:2,000 to about 1:100,000 by volume.

31. The method of claim 30, wherein in step (d) the ratio of the diluted non-aqueous liquid composition to the aqueous dissolution medium is from about 1:5,000 to about 1:40,000 by volume.

32. The method of claim 1, wherein the dissolution testing apparatus is a paddle assembly.

33. Method of determining the dissolution rate of an analyte in a non-aqueous liquid composition, comprising the steps of:
(a) providing a non-aqueous liquid composition comprising an analyte and a non-aqueous base;
(b) introducing at least part of the non-aqueous liquid composition and an aqueous dissolution medium into a dissolution testing apparatus, wherein the aqueous dissolution medium comprises a buffer having a molarity of from about 1 mM to about 10 mM;
(c) contacting the non-aqueous liquid composition and the aqueous dissolution medium for a predetermined time; and
(d) determining the amount of analyte in the aqueous dissolution medium.

34. Method of determining the dissolution rate of an analyte in a non-aqueous liquid composition, comprising the steps of:
(a) providing a non-aqueous liquid composition comprising an analyte and a non-aqueous base;
(b) introducing at least part of the non-aqueous liquid composition and an aqueous dissolution medium into a dissolution testing apparatus, wherein the volume ratio of non-aqueous liquid composition to aqueous dissolution medium in the dissolution testing apparatus is from about 1:2,000 to about 1:100,000;
(c) contacting the non-aqueous liquid composition and the aqueous dissolution medium for a predetermined time; and
(d) determining the amount of analyte in the aqueous dissolution medium.
